(19) United States
(12) Patent Application Publication
(10) Pub. No.: US 2023/0060532 A1
(43) Pub. Date: Mar. 2, 2023

(54) METAL-ORGANIC FRAMEWORK COATED ORGANIC FIELD EFFECT TRANSISTOR BASED NO2 SENSOR AND METHOD

(71) Applicant: KING ABDULLAH UNIVERSITY OF SCIENCE AND TECHNOLOGY, Thuwal (SA)

(72) Inventors: Valeriya CHERNIKOVA, Thuwal (SA); Mohamed EDDAOUDI, Thuwal (SA); Khaled Nabil SALAMA, Thuwal (SA); Osama SHEKHAH, Thuwal (SA); Sandeep G. SURYA, Thuwal (SA); Mani Teja VIJJAPU, Thuwal (SA); Saravanan YUVARAJA, Thuwal (SA)

(21) Appl. No.: 17/796,933

(22) PCT Filed: Feb. 1, 2021
(86) PCT No.: PCT/IB2021/050804

§ 371 Date: Aug. 2, 2022

Related U.S. Application Data
(60) Provisional application No. 62/969,255, filed on Feb. 3, 2020.

(51) Int. Cl. G01N 27/414; G01N 33/00

(52) U.S. Cl. CPC G01N 27/4141; G01N 33/0037; B01J 20/226

(57) ABSTRACT

An NO₂ detection device includes a substrate; a drain formed on the substrate; a source formed on the substrate; a p-type polymer semiconductor layer formed on the substrate, between the drain and the source; and an n-type metal-organic framework layer located over the p-type polymer semiconductor layer. The n-type metal-organic framework layer has apertures having a size larger than a size of the NO₂ molecules so that the NO₂ molecules pass through the n-type metal-organic framework layer to arrive at the p-type polymer semiconductor layer to increase an electrical current.

This application claims priority to U.S. Provisional Patent Application No. 62/969,255, filed on Feb. 3, 2020, entitled “SYNERGISTIC COMBINATION OF PDVT-10 AND PORPHYRIN-MOF FOR AN ULTRASENSITIVE AND HIGHLY SELECTIVE NO2 OFET SENSOR,” the disclosure of which is incorporated herein by reference in its entirety.

### BACKGROUND

### Technical Field

Embodiments of the subject matter disclosed herein generally relate to a NO₂ sensor and method, and more particularly, to an organic transistor that uses a donor-acceptor conjugated polymer coated with a metal-organic framework (MOF) for detecting the presence of the NO₂ gas.

### Discussion of the Background

Air pollution is one of the most serious problems faced by the population all around the world. One of the dangerous gasses that contributes to the air pollution and is detrimental to human beings and environmental resources is nitrogen dioxide (NO₂). According to an Occupational Safety and Health Administration (OSHA) report, the short time exposure limit (STEL) to NO₂ gas for healthy subjects is 1 ppm for 15 minutes. Thus, a sensor to detect NO₂ gas molecules over a wide range of concentrations with a high precision, resolution, and accuracy is desired.

Several detection techniques exist for gas sensing strategies, such as the electromagnetic spectroscopic method, optical fiber, electrochemical, quartz crystal microbalance (QCM), microelectromechanical systems (MEMS), resistance changes, chemi-capacitive, mass spectrometry, and surface potential measurement. Some of the drawbacks of the above techniques are their bulk designs, environmental interferents, high-operating temperature, and power consumption. As an alternative to silicon dominated electronics (complementary metal oxide semiconductor, or CMOS), recently, many research groups have explored the potential of emerging organic and flexible platforms for electronic devices in environmental sensing applications [1-4]. In the field of gas sensors, organic field effect transistor devices are preferred due to the ease of their solution process and reduced device complexity when compared to CMOS technology [1]. However, organic semiconductors (OSC) also have some serious problems, such as poor stability and low charge carrier mobility, when compared to the inorganic semiconductors [1, 2, 5].

To overcome these issues, donor-acceptor (D-A) based conjugated polymers are considered as alternatives due to their good electronic properties and improved stability [6-10]. A rapidly emerging D-A conjugated polymer that is at forefront is the Diketopyrrolopyrrole (DPP) copolymer, which has been successfully explored for various sensing activities [11-16]. Similarly, conjugated thiophenes (CTs) have also been examined for explosive detection, where CT acts as a donor block for electron deficient explosive molecules [16-17]. However, no NO₂ sensing device has been proposed to use the stable D-A conjugated polymer based OFET device.

Thus, there is a need for a new NO₂ sensor that is highly sensitive and selective for gas sensing applications and overcomes the above discussed limitations of the existing sensors.

### BRIEF SUMMARY OF THE INVENTION

According to an embodiment, there is an NO₂ detection device that includes a substrate, a drain formed on the substrate, a source formed on the substrate, a p-type polymer semiconductor layer formed on the substrate, between the drain and the source, and an n-type metal-organic framework layer located over the p-type polymer semiconductor layer. The n-type metal-organic framework layer has apertures having a size larger than a size of the NO₂ molecules so that the NO₂ molecules pass through the n-type metal-organic framework layer to arrive at the p-type polymer semiconductor layer to increase an electrical current.

According to another embodiment, there is an n-type metal-organic framework material that includes [M′₂L₂(M″F₆)]ₙ, wherein M′ is a metal with octahedral geometry, L is ditopic nitrogen containing linker, and (M″F₆) is an inorganic pillar.

According to yet another embodiment, there is a method of making an NO₂ detection device, and the method includes dissolving a p-type polymer semiconductor material (PDVT-10) into a solvent, generating an n-type metal-organic framework material (MOF-A), providing a substrate based on Si, forming a drain and a source on the substrate, depositing the p-type polymer semiconductor material (PDVT-10) onto the substrate, between the drain and the source, to form a polymer semiconductor layer, and depositing the n-type metal-organic framework material (MOF-A) onto the polymer semiconductor layer to form an n-type metal-organic framework layer. The n-type metal-organic framework layer has apertures having a size larger than a size of the NO₂ molecules so that the NO₂ molecules pass through the n-type metal-organic framework layer to arrive at the p-type polymer semiconductor layer to increase an electrical current.

### BRIEF DESCRIPTION OF THE DRAWINGS

For a more complete understanding of the present invention, reference is now made to the following descriptions taken in conjunction with the accompanying drawings, in which:

FIG. 1 is a schematic diagram of a NO₂ detection device;

FIG. 2 is a schematic diagram of another NO₂ detection device that uses a combination of a p-type polymer semiconductor layer (PDVT-10) and an n-type MOF material (MOF-A);

FIG. 3 is a schematic diagram of the NO₂ detection device of FIG. 2, which illustrates how the MOF-A material acts as a sieve;

FIG. 4 illustrates the Raman spectrum of the polymer semiconductor layer for the spectral range from 1000 to 1700 cm⁻¹ and (inset) 2700 to 3000 cm⁻¹;

FIG. 5 is a table showing the Raman Shift peaks of the PDVT-10 organic semiconductor;

FIG. 6 is a table illustrating the electronic band structure values of the PDVT-10 and MOF materials;

FIG. 7 illustrates the structural composition of the MOF-A material and its precursor;

FIG. 8 illustrates a stability test of the MOF-A material upon activation and exposure to air for 96 hours;

FIG. 9 illustrates an estimate of the adsorption capacity of the material at 500 ppm NO₂ concentration;

FIGS. 10A to 10C illustrate the transfer characteristics, output behavior, and selectivity of the NO₂ detection device of FIG. 1;

FIG. 11 presents the transistor parameters of the devices shown in FIGS. 1 and 2;

FIGS. 12A to 12C illustrate the transfer characteristics, output behavior, and selectivity of the NO₂ detection device of FIG. 2;

FIG. 13 shows the relation between the current ratio (CR) and subthreshold swing (SS) for different NO₂ gas concentrations from 1 to 100 ppm;

FIG. 14 illustrates the current response behavior of the device of FIG. 2 for a wide range of NO₂ gas concentrations from 25 ppb to 50 ppm;

FIG. 15 illustrates the response and recovery characteristics of the device of FIG. 2 for 25 ppb NO₂ gas;

FIG. 16A schematically illustrates an interaction of the NO₂ gas with the device of FIG. 1, FIG. 16B illustrates a heterojunction structure of the device of FIG. 2, and FIG. 16C illustrates an interaction of the NO₂ gas with the device of FIG. 2;

FIG. 17 is a flow chart of a method for making the device of FIG. 2; and

FIG. 18 is a flow chart of a method for measuring the concentration of NO₂ with the device of FIG. 2.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a heterojunction made of a highly porous specific metal-organic framework (MOF) and a D-A conjugated polymer in an organic field effect transistor (OFET). However, the embodiments to be discussed next are not limited to the specific MOF or D-A conjugated polymer discussed herein, but may be applied to other combinations of similar elements.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel NO₂ gas sensor is using a new variant of the DPP copolymer with thiophene donor blocks (called herein PDVT-10) as a channel layer in an OFET device. The PDVT-10 is an air-stable polymer of p-type nature with majority carriers as holes. The (E)-2-(2-(thiophen-2-yl)vinyl) thiophene (TVT) functional unit in the PDVT-10 is an electron donor. Thus, any oxidation process with this material will lead to an increase in the concentration of the majority carriers, thereby increasing the electrical conduction. The fact that this polymer has a lower highest occupied molecular (HOMO) level makes it a stable counterpart for similar materials like P3HT.

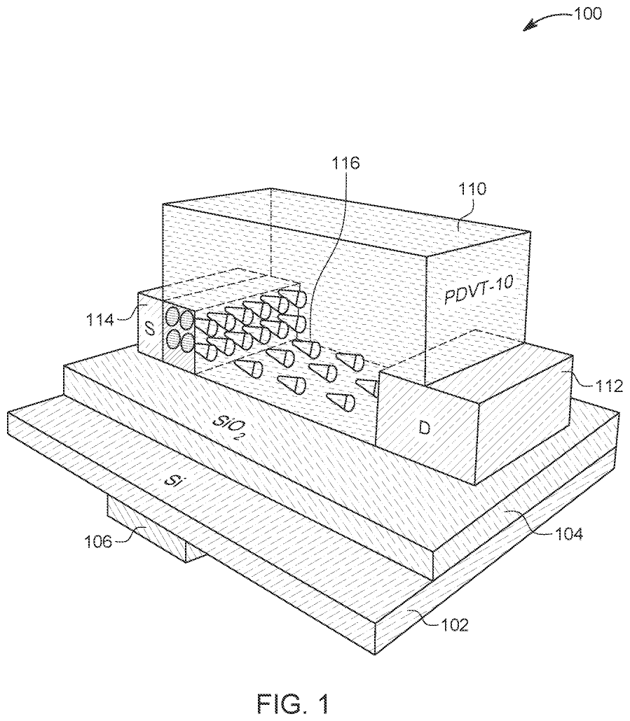

Thus, the PDVT-10 having the above features serves as an organic semiconductor, which stands as a good candidate for selective detection of an electrophilic analyte such as the NO₂ gas. A bottom-gate bottom-contact (BGBC) topology of pristine PDVT-10 OFET 100, which is illustrated in FIG. 1, exhibits a low-threshold voltage, a subthreshold swing, and a high-charge carrier mobility. The pristine PDVT-10 OFET 100 is shown in FIG. 1 as being formed on a Si substrate 102, that has a top SiO₂ film 104. The PDVT-10 material 110 is formed on the SiO₂ film 104, to be sandwiched between a drain 112 and a source 114. The positive carriers 116 are schematically illustrated as moving from the source 114 to the drain 112. The pristine OFET device 100 displays appreciable selectivity, but poor sensitivity towards the NO₂ gas molecules. An optional electrode 106 may be formed directly on the Si substrate 102 to act as a gate.

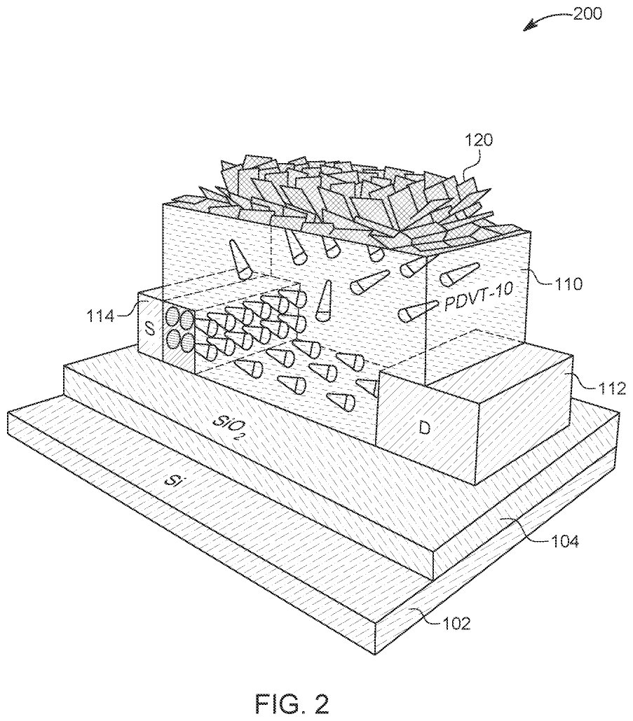

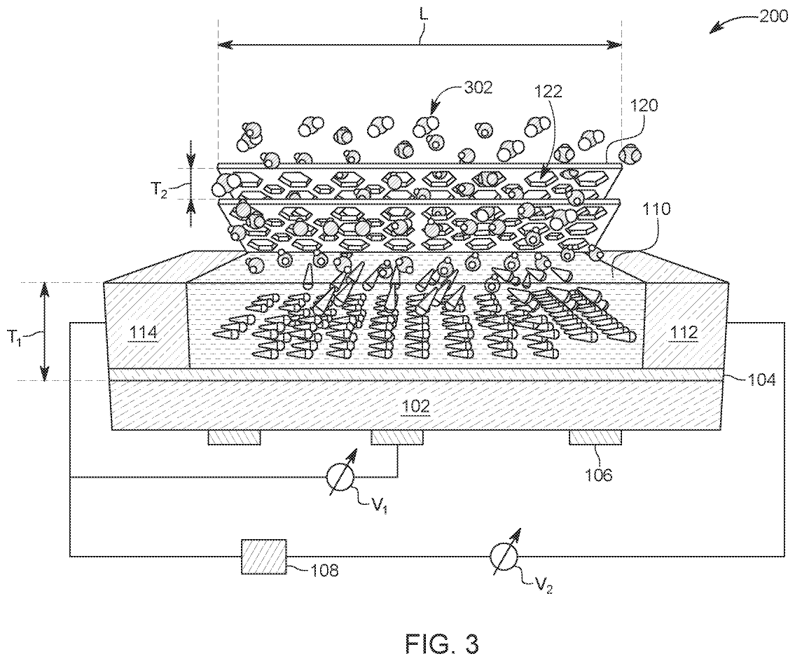

To improve the sensitivity of the pristine OFET device 100, a heterojunction combination of a porous n-type MOF with the PDVT-10 polymer is proposed herein, as illustrated in FIG. 2. The novel heterojunction based OFET device 200 has the PDVT-10 polymer layer 110 covered (e.g., coated) with a layer 120 of the MOF material. The quest for room-temperature stable and sensitive gas sensors has motivated researchers to contemplate alternative materials such as MOFs due to their exceptional properties in terms of reversible physisorption and highly-accessible pore system, prompting effective and selective interactions with the targeted analytes. This sensing can be achieved using different transduction mechanisms, including capacitive, mass, and frequency changes. The selectivity of the MOF materials for targeted analytes is highly desirable, but still not well developed.

Molecular sieving is among the possible mechanisms used to introduce molecular selectivity. Another source of selectivity is the specific chemical interactions between the adsorbate/analyte with the MOF internal surface, via hydrogen bonding, or other types of interaction. In such mechanisms, the atoms or molecules that are smaller in size than the MOF's apertures can diffuse inside, but larger molecules will be rejected. This principle is illustrated in FIG. 3, which shows the heterojunction based OFET device 200 and the apertures 122 of the MOF layer 120. The NO₂ gas molecules 302 have a diameter smaller than the diameter of the apertures 122, and thus, these molecules move through the MOF layer 120 and arrive at the PDVT-10 material 110. However, the molecules having a size larger than the size of the apertures 122 are prevented from arriving to the PDVT-10 material 110. To ensure this selectivity, in one embodiment, as illustrated in FIG. 3, the drain 112, source 114, and the MOF layer 120 fully encompass the PDVT-10 material 110 so that only molecules or atoms from the ambient that can fit through the apertures 122 can reach the PDVT-10 material 110.

In one application, a length L of the PDVT-10 layer is about 10 μm, a thickness T1 of the PDVT-10 layer is between 15 and 70 nm, and a thickness T2 of the MOF-A layer is between 10 and 100 nm. In a preferred embodiment, T1 is 20 nm and T2 is 20 nm. FIG. 3 also shows a first power source V1 being coupled between the source and gate and a second power source V2 being coupled between the source and the drain. In one application, a single power source may be used to generate the two voltages that are otherwise generated by the sources V1 and V2. Electronics 108 may be used to determine a drain current.

Different types of MOF materials can be used as a selective sensing layer for detecting different gases and VOCs using different transduction mechanisms. For example, it is possible to use a three dimensional (3D) MOF described by [M′₂L₂(M″F₆)]ₙ, where M′ is a metal with octahedral geometry, L is ditopic nitrogen containing linker, and (M″F₆) is an inorganic pillar. These materials have shown encouraging properties in terms of gas separation and sensing applications. The structure of this type of MOF is inherently modular, as each of all three building components can be substituted with a variety of chemical entities and combined in a practically unlimited number of combinations to suit various purposes.

The inventors have explored the limited number of MOFs based on tetratopic ligand and hexafluorometalates -[M′L(M″F₆)]ₙ [19-21]. In this embodiment, a MOF based on 5,10,15,20-Tetra(4-pyridyl)porphyrin (TPyP) linker and Ni(TiF₆) inorganic pillar was synthesized and chosen due to its thermal stability at normal relative humidity. The development of stable microporous compounds based on porphyrin building blocks is desired due to the variety of functionalities such building blocks can offer. Porphyrins are valued for their unique light-harvesting, optical response, sensing, and catalytic properties.

Thus, according to an embodiment, a MOF-A (MOF based on tetratopic ligand and hexafluorometalates) was applied as a coating over the layer 110 of PDVT-10 for sensing the NO₂ gas. In this embodiment, the employed MOFs improved the sensitivity of the device 200 by 700% relative to the device 100, without compromising the selectivity behavior towards the NO₂ analyte. In addition, the sensing properties of the MOF-A/PDVT-10 device 200 proved to be highly stable against relative humidity. A sensing mechanism is discussed that explains the interaction between the device stack and the NO₂ gas analytes. To the inventors' knowledge, this is the first time a DPP based OFET sensor has been reported for the detection of NO₂ gas with high sensitivity and reasonable selectivity (both oxidizing and reducing gases).

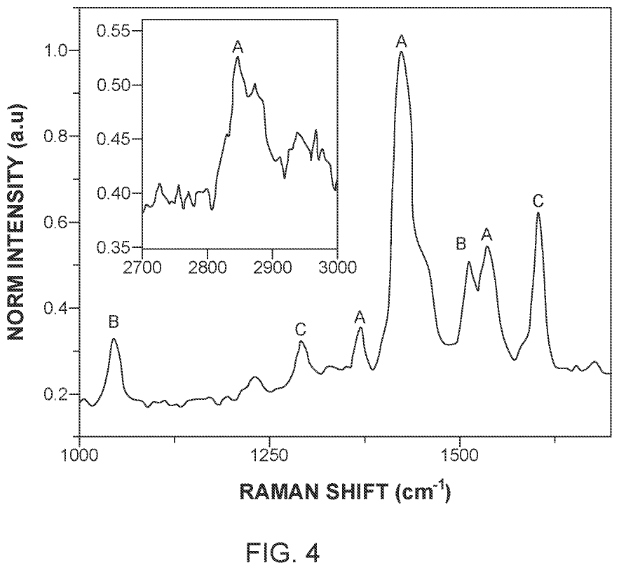

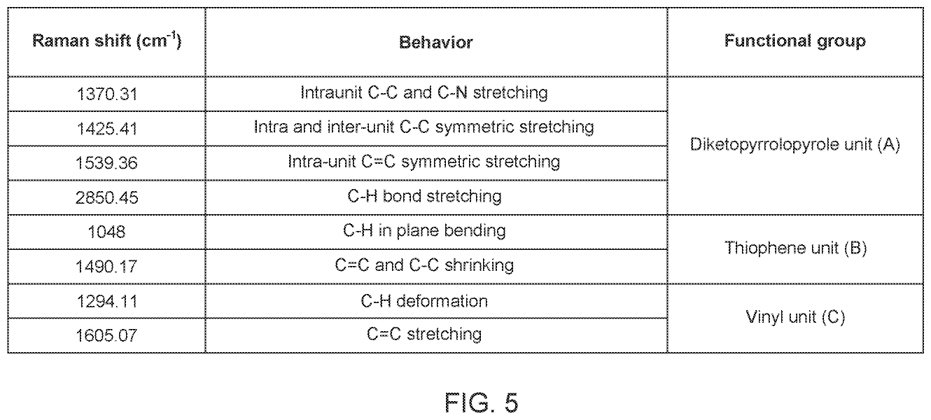

The novel BGBC MOF-A/PDVT-10 OFET sensor 200 was characterized for various parameters as now discussed. The organic semiconductor PDVT-10, which consists of DPP and Thiophene-Vinyl-Thiophene (TVT) acting as electron acceptor and donor units, respectively, was observed with the help of a Raman spectrum at an excitation wavelength of 473 nm, as shown in FIG. 4. This spectrum consists of 10 peaks associated with three polymer units, as illustrated in the table in FIG. 5. A spin coating process was used for the deposition of the PDVT-10 polymer layer 110 over the surface of the SiO₂ layer 104. In one application, a uniform and conformal film was deposited as the layer 110, with a thickness of around 17 nm. Furthermore, with the help of atomic force microscopy (AFM) measurement, the roughness of the PDVT-10 thin film was measured to be around 0.9 nm.

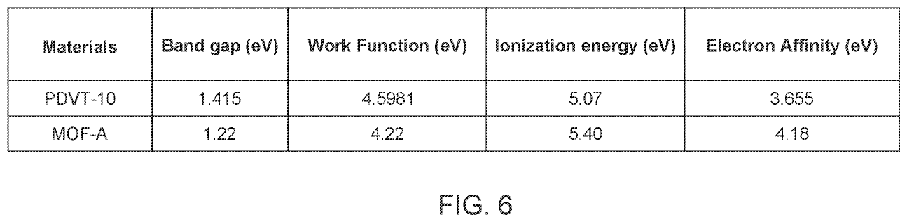

The work function (ϕ) of the PDVT-10 layer has been determined. The work function of a material is defined as the difference between vacuum level and fermi energy. The work function ϕ₁ was quantitatively determined for the PDVT-10 layer to be 4.5981 eV, with the help of Kelvin probe force microscopy (KPFM). The KPFM helped to determine the electronic band gap of the PDVT-10 material along with the ionization potential and electron affinity values. Besides the work function, the ionization energy or HOMO level of the polymer was measured to be around 5.07 eV, which was determined using photoelectron yield spectroscopy in air (PYS). This was followed by the UV-Vis-IR absorption spectroscopy technique, which was employed to observe the optical absorption behavior and optical band gap of a polymer. The obtained absorption spectrum exhibited a maximum peak at 715 nm accompanied by a shoulder peak at 789.5 nm, which closely matches the reported PDVT-10 spectrum. The optical band gap of the polymer was approximately 1.415 eV; it was extracted from the Tauc plot of the UV-Vis-IR absorption spectrum shown. This value is close to the values reported in the literature. From the table shown in FIG. 6, it can be seen that the work function of the polymer is near its ionization energy level. This shows that the polymer is a typical p-type organic semiconductor.

The synthesis of the MOF-A layer 120, i.e., the [Ni(TPyP)(TiF₆)]ₙ material, involved the generation of MOF-A particles, which were then coated on the polymer layer 110. The synthesis of the MOF-A particles was carried out under ultrasonic irradiation at a frequency of 40 KHz and 60° C. preset in an ultrasonic bath. In one application, the Ni(NO₃)₂.6H₂O (0.04 mmol) and TPyP (0.02 mmol) were partially dissolved in 6 mL of DMF, and later 0.4 mL of 0.05M aqueous solution of (NH₄)₂TiF₆ (0.02 mmol) was added into a 20 mL scintillation vial. The vial was placed in a pre-heated ultrasonic bath for 10 hr. A clear color change of the initial dispersed phase from dark purple to bright purple indicated the transformation of the reagents. The product was isolated by centrifugation, and it was washed multiple times with DMF and activated via solvent exchange with methanol. The formed [Ni(TPyP)(TiF₆)]ₙ MOF-A was stable under ultrasound irradiation, which allowed to effectively separate the product from dissolving starting materials by performing the extensive washing in the sonication bath.

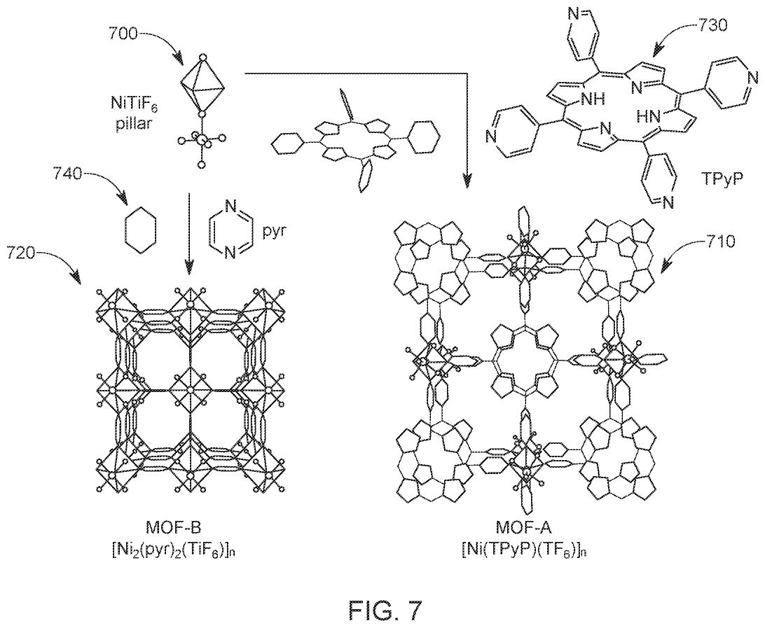

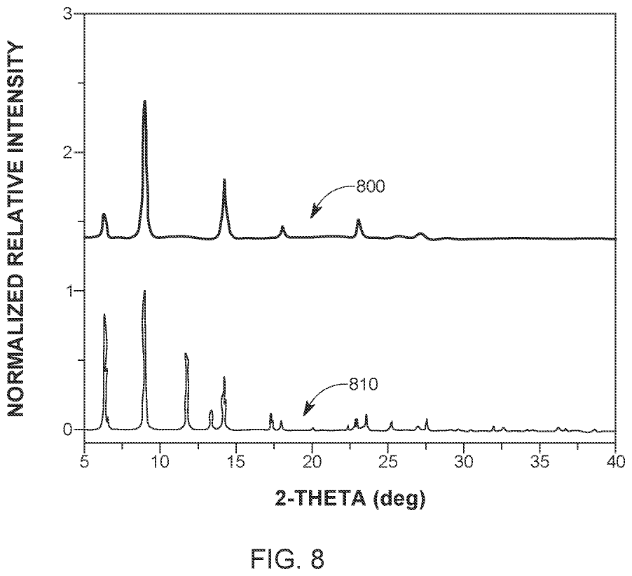

FIG. 7 illustrates the structure of the chosen MOF (MOF-A) 710 and its ancestor (MOF-B) 720, which was accessed by using the tetratopic square ligands, namely TPyP 730 instead of ditopic (pyrazine (pyr), 4,4′-dipyridine (dpy)) 740. It is noted that both the MOF-A 710 and the MOF-B 720 are generated from NiTiF₆ pillars 700. The PXRD analysis shown in FIG. 8 confirms the formation and phase purity of the MOF-A 710, via the excellent match between the experimental 800 and simulated 810 patterns. MOF-A adsorbs N₂ with a characteristic for microporous materials with fully reversible Type-I isotherms. The apparent BET surface area for heated MOF-A particles was estimated to be 991 m²/g, and a pore volume of 0.49 cc/g was projected at 0.85 relative pressure, as the increase of the uptake from 0.9 to 1 relative pressure is associated with the small size of the particles. The estimated pore volume is in good agreement with the theoretical value of 0.49 cc/g.

Typically, sonochemical synthesis shortens the synthesis time and helps to reduce the size of the crystals compared to a typical solvothermal synthesis method. The same was found for the MOF-A. The inventors obtained MOF-A having approximately 1 μm by 1 μm square plate-like crystals with a thickness in the range of tens of nanometers. In addition, the elemental analysis showed the presence of expected elements and the right atomic ratio of Ni and Ti in the structure. This resulting morphology of the synthesized crystals allows them to be easily dispersed and spin coated as a thin film on different supports, e.g., the PDVT-10 layer 110.

The stability at normal relative humidity was confirmed by the exposure of the MOF-A particles to the normal lab environment for 2 weeks (20° C. and 45% RH). To understand the thermal stability of the MOF-A material, the same MOF-A particles (used in a relative humidity experiment) were heated to 200° C. in the presence of air for more than 96 hours. The porosity of the heated sample did not change and was similar to the synthesized one, which confirms the thermal stability of MOF-A. Furthermore, the UV-VIS spectra confirmed that the MOF-A was not fully metallated during synthesis due to the presence of some of the characteristic peaks of the nonmetallated ligand.

Similar to the PDVT-10 material, some of the electronic properties of the MOF-A particles, such as band gap, ionization energy, and electron affinity, were experimentally obtained and the corresponding parameter values are displayed in the table in FIG. 6. The n-type behavior of the MOF-A was confirmed using the work function, which stays very close to the electron affinity level. On the one hand, when compared to the PDVT-10 material, due to their high electron affinity, the MOF particles inhibit the tendency to attract electrons from the underlying PDVT-10 layer. On the other hand, higher ionization energy supports the stability of the MOF particles, which is in line with the previously discussed XRD results.

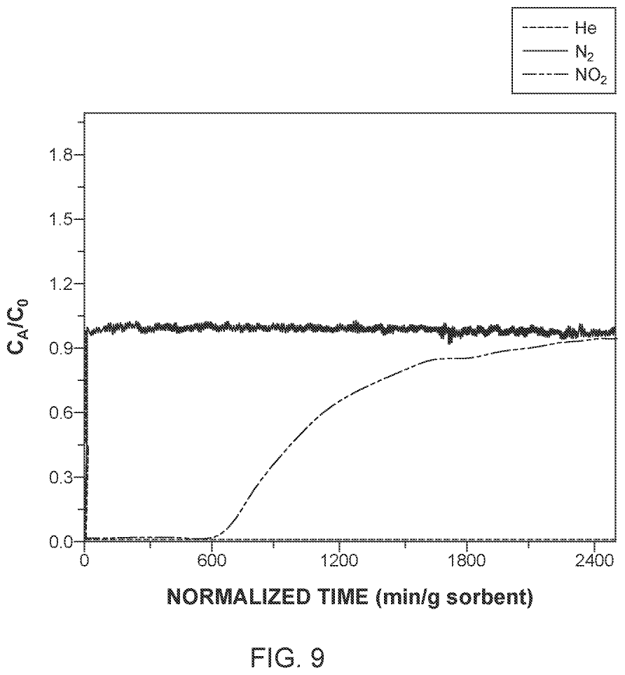

To study the interaction of the MOF-A layer 120 with the NO₂ molecules, the following experiment was carried out with 420 mg of an activated sample using a 500 ppm NO₂ in N₂ gas mixture with a 50 cc/min flowrate at 298 K and 1 bar. FIG. 9 displays the normalized retention time for NO₂ 600 min/g, which shows a high affinity of the MOF-A to NO₂, corresponding to an uptake of 0.66 mmol/g at 500 ppm NO₂ concentration.

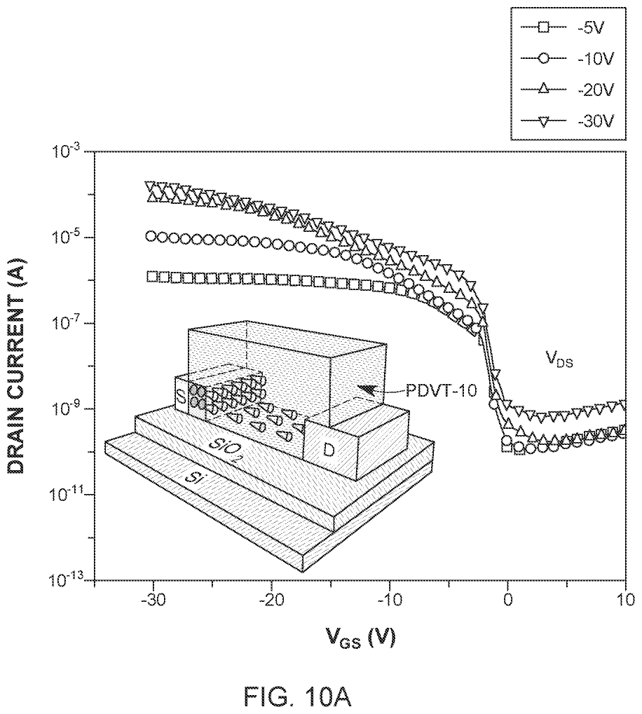

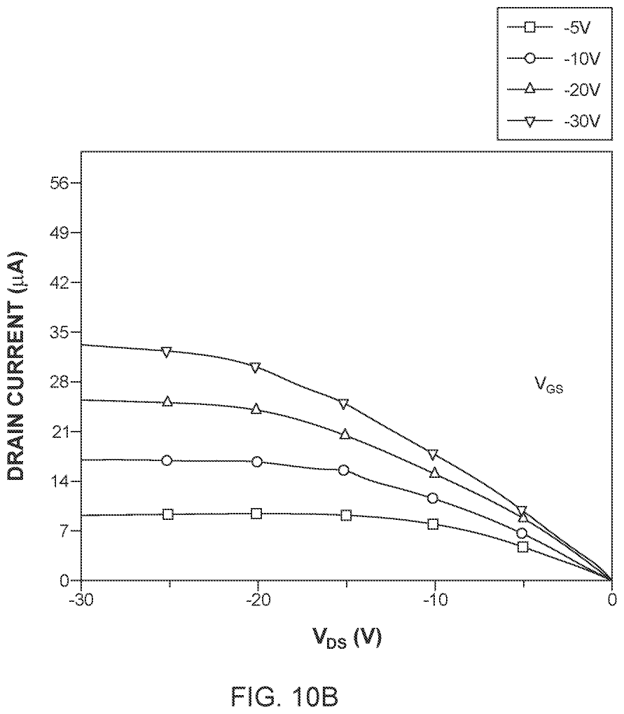

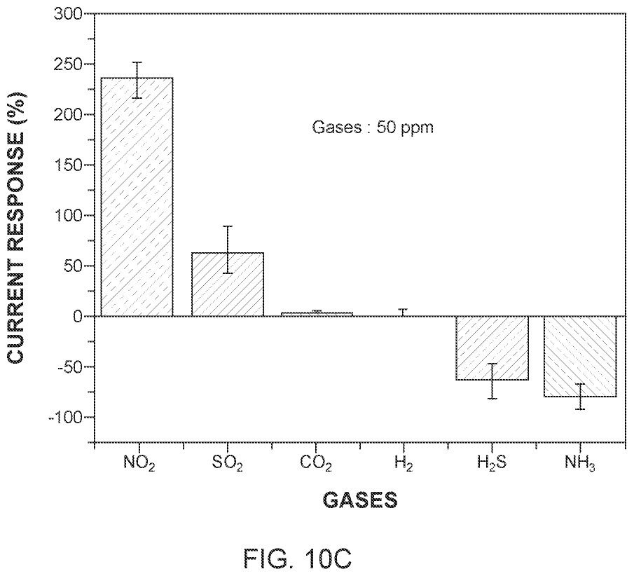

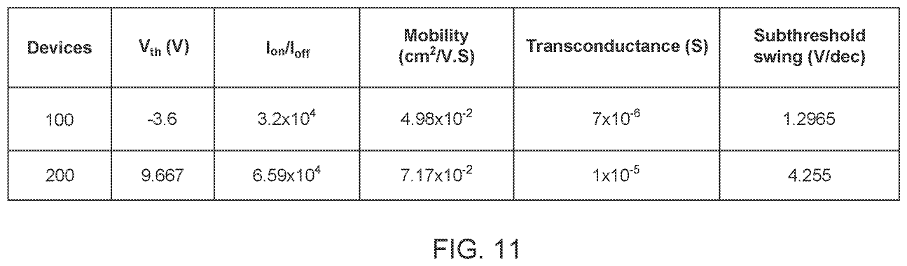

The electrical and gas sensing performance of the OFET device 200 was next studied. The OFET devices 100 and 200 with a BGBC geometry were fabricated on an Si/SiO₂ substrate using a standard CMOS compatible process. Interdigitated source and drain contacts 112 and 114 were patterned on the surface of the Si/SiO₂ substrate 102/104 using a photolithography process. For the pristine PDVT-10 OFET device 100, the polymer film 110 was deposited on top of the substrate patterned with the source and drain electrodes 112 and 114. The formation of an ultrathin channel layer 110 is one of the desired features to improve the interaction with the gas analytes at the dielectric/semiconductor interface. As shown in FIG. 10A, to obtain the transfer characteristics, the device 100 was subjected to a voltage bias by sweeping the gate voltage (VG) from +20 to −30 V for different drain voltage steps (VD) from −5V to −30V. Note that the gate voltage is applied to the gate 106 while the drain voltage is applied to the drain 112 by an external voltage source (not shown). It is seen in FIG. 10A that the device 100 turns “ON” in the negative gate bias region, essentially confirming that the PDVT-10 layer 110 is a p-type organic semiconductor. The transistor parameters such as threshold voltage (Vₜₕ), charge carrier mobility (μ), subthreshold swing (SS), and current ratio (I_{ON}/I_{OFF}) were extracted from the transfer characteristics of the device and their values are tabulated in table in FIG. 11. It is observed a low threshold voltage in the device 100, which can be attributed to the low potential trap states at the semiconductor/dielectric interface region. FIG. 10B illustrates the output characteristics of the device 100 obtained by sweeping the drain voltage from 0 to −30V with gate bias from −5V to −30V. The outcome mimics the nearly ideal output behavior of a transistor with a good linear and saturation regimes, and thus these devices were investigated for gas sensing application. However, the selectivity of the device 100, which is illustrated in FIG. 10C is poor, as the current response of the device (which is plotted on the Y axis) is below 250% for the studied gases.

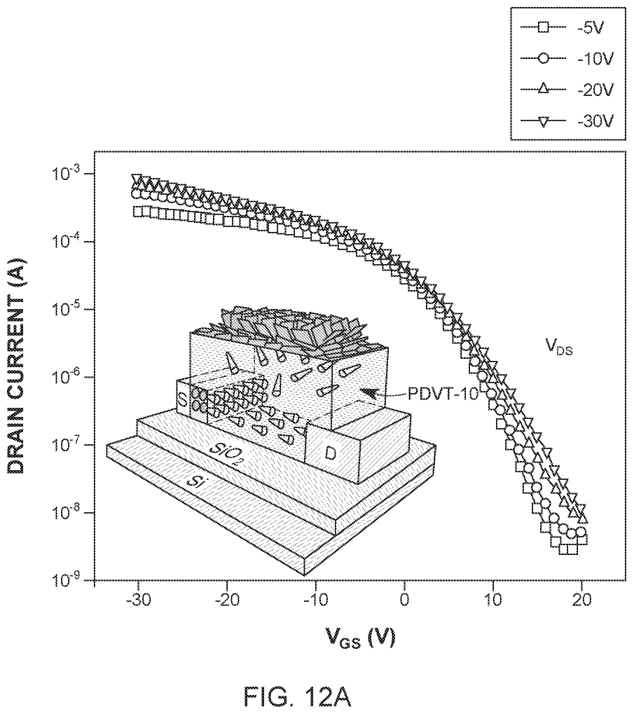

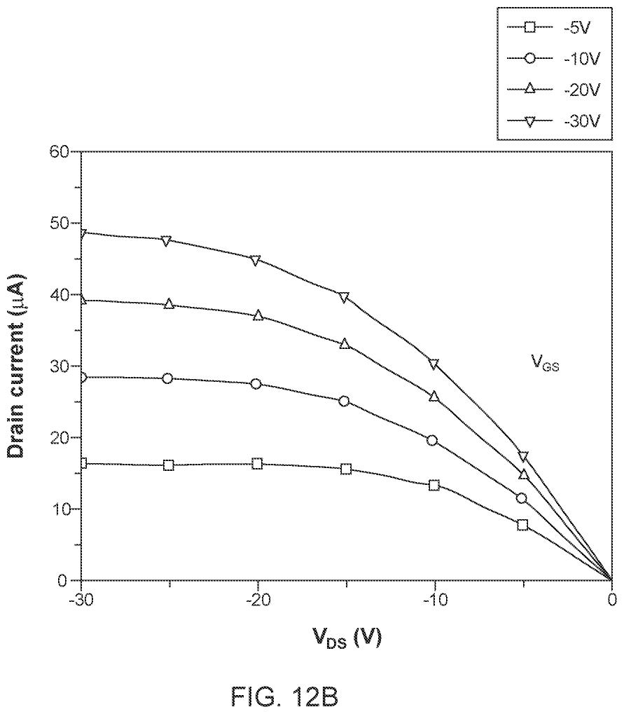

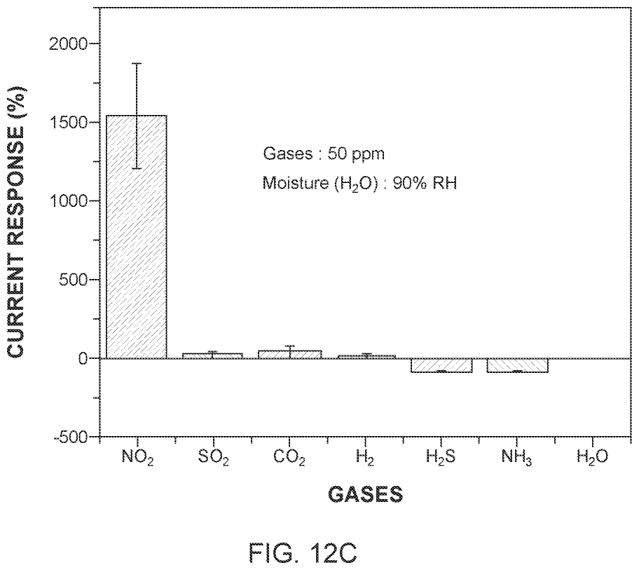

Similarly, the MOF-A/PDVT-10 OFET device 200 was fabricated and characterized to understand the effect of the porous MOF-A on the device's characteristics and gas sensing performance. The fabrication process for both devices 100 and 200 was the same until the formation of the polymer channel layer. To complete the MOF-A/PDVT-10 device 200's fabrication process, an additional step was the drop casting of the MOF-A particles on the surface of the polymer film 110. The biasing conditions used to obtain the transfer and output characteristics of the MOF-A/PDVT-10 device 200 remained the same as those of their pristine device counterpart. In device 100, because the active material is a p-type organic semiconductor, the negative threshold voltage was observed at around −3.6 V. With the addition of the MOF-A particles, as presented in FIG. 12A, a significant positive threshold voltage shift took place from −3.6 V to +9.667 V, as mentioned in the table of FIG. 11. This was followed by the change in the charge carrier mobility of the MOF-A/PDVT-10 device 200 to 7.17×10⁻² cm²N*s, which is around 43% higher than that of the device 100. The positive shift of the threshold voltage and the increase in the charge carrier mobility are attributed to the accumulation of excess charge carriers in the formed channel region. This behavior may be the result of the charge transfer process between the PDVT-10 and MOF-A layers, which is discussed later.

The current ratio (I_{ON}/I_{OFF}) is an important parameter for defining the performance of the desired OFET device. As shown in the table of FIG. 11, the current ratios of devices 100 and 200 are 3.2×10⁴ and 6.59×10⁴, respectively. Compared to the device 100, the MOF-A/PDVT-10 device 200 exhibited good output behavior with an increase in the drain current level for different gate voltages, as seen in FIG. 12B. As a result, the improved charge carrier mobility, transconductance, and positive threshold voltage shift of the MOF-A/PDVT-10 device 200 led to a better device performance when compared to its pristine counterpart 100. In this regard, it is noted that FIG. 12C shows the current response of the MOF-A/PDVT-10 device 200 being about 6 times better for the NO₂ gas then the current response of the device 100 (see FIG. 10C).

The selectivity behavior of both the device 100 and MOF-A/PDVT-10 device 200 were evaluated by recording their drain current responses towards both oxidizing and reducing gases at a fixed concentration of 100 ppm. Current responses were obtained from the transfer characteristics of the devices by sweeping VG from −20 V to +30 V at constant −20 VD. Among all the studied gases, the device 100 showed a good sensitivity of about 225%, and an increase in drain current towards 100 ppm NO₂ gas. As a result, the device 100 displayed moderate sensitivity with good selectivity towards the targeted NO₂ gas. The same set of gases was employed to study the selectivity of the MOF-A/PDVT-10 device 200. It was observed that its sensitivity towards 100 ppm NO₂ was around 3 orders of magnitude greater than its pristine counterpart. Thus, the MOF-A layer plays a significant role in augmenting the sensitivity of the device without influencing the selective nature of the pristine device 100 towards the targeted NO₂ gas.

The MOF-A/PDVT-10 device 200 was exposed to different gas concentrations from 0 to 50 ppm, and the corresponding transfer characteristics were obtained by sweeping the gate voltage from +30 to −20 V at fixed −20 V drain bias. From the obtained transfer characteristics, a significant positive threshold shift from ˜−5 V to −20 V was witnessed, and the drain current increased from 0.1 mA to 1.1 mA with the gas concentrations. Sequentially, the output characteristics of the MOF-A/PDVT-10 device 200 towards different gas concentrations (0 to 50 ppm) were obtained by biasing the device with a drain voltage from 0 to −30 V at fixed −20 VG. For the concentrations above 20 ppm, the drain current exhibited a non-saturation regime for all biases. This can be attributed to the transfer of excess charge carriers in the channel region, preventing the device current from reaching saturation.

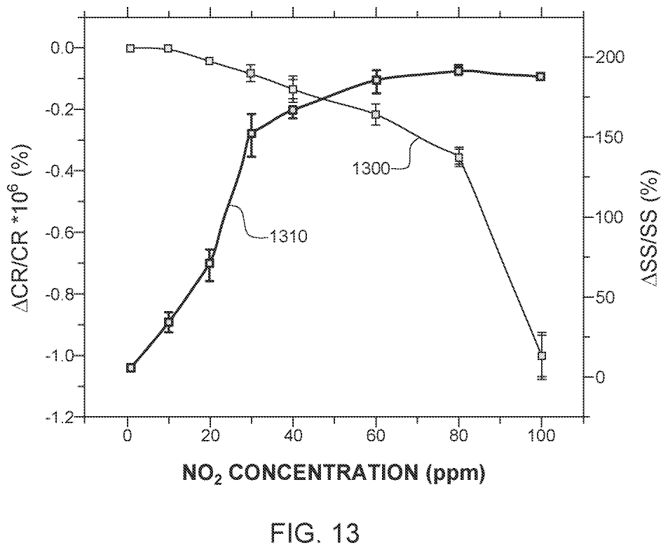

Using the obtained transfer characteristics, the effect of the gas concentrations on the transistor parameters of the device were explored. As the concentrations increased from 0 to 100 ppm, there was a decrease in the threshold voltage of around 3 orders of magnitude. In contrast, the charge carrier mobility increased linearly with gas concentration. As a result, the measured percentage change of charge carrier mobility at 100 ppm was around 400%. FIG. 13 displays the obtained relationship 1300 between the current ratio (CR) change and different gas concentrations. From this figure, it is noted that the current ratio decreased exponentially with the gas concentrations from 1 to 100 ppm. The current ratio of the MOF-A/PDVT-10 device 200 at 100 ppm NO₂ gas was about 6 orders of magnitude smaller than the response under N₂ ambient conditions.

Another parameter of a transistor device is the subthreshold swing (SS), and this parameter was studied with various gas concentrations. It was found that the SS was inversely proportional to the charge carrier trap density at the dielectric/semiconductor interface (in the concentration range from 1 to 15 ppm), after which the SS tends to saturate as the traps were quenched, as also illustrated in FIG. 13 by curve 1310. Moreover, the reduction of trap/defect states at the interface not only lowered the threshold voltage but also augmented the charge carrier mobility of the MOF-A/PDVT-10 device 200, which is in line with the previous results.

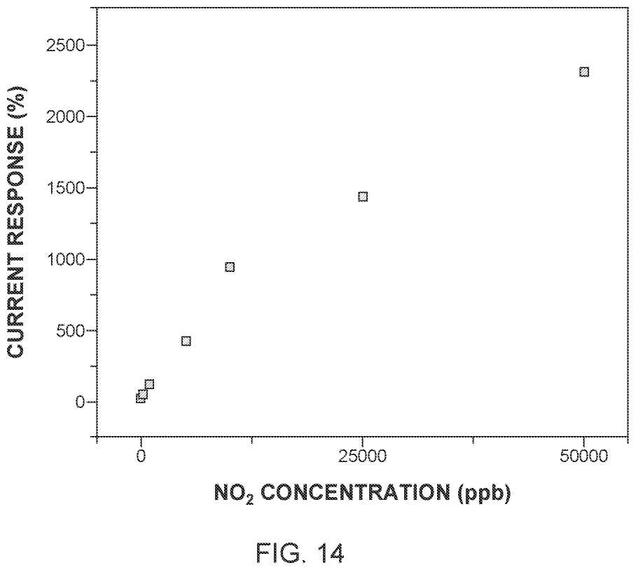

Some other parameters, such as the limit of detection, response, and recovery times, were extracted from the transient analysis of the MOF-A/PDVT-10 device 200 in the presence of the NO₂ analyte. The transient response was obtained from the MOF-A/PDVT-10 device 200 for VD=VG=−20 V under the exposure at NO₂ concentrations from 25 ppb to 50 ppm for a period of 5 min each. After each exposure cycle, the device was allowed to recover until the drain current approached the base level. It was observed that with the increase in the gas concentration, the probability of the device for a complete recovery gradually decreased. In this respect, FIG. 14 displays the percentage change in the drain current response towards different gas concentrations. At 50 ppm gas exposure, the drain current (%) of the MOF-A/PDVT-10 device 200 was increased by 3 orders of magnitude. The sensitivity of the MOF-A/PDVT-10 device 200 to the NO₂ gas was found to be approximately 680 nA ppb⁻¹. The detection limit of the device was calculated by the root mean square deviation (RMSD) method, which consists of 3 data points from 25 ppb to 250 ppb. Using the RMSD method, the limit of detection (LOD) was calculated to be around 8.25 ppb, which is much lower than the LOD values for NO₂ reported in the literature. Furthermore, the MOF-A/PDVT-10 device 200 exhibited high stability against strong relative conditions with a sensitivity of around 0.005% RH⁻¹, thus outperforming any reported device to date. The reproducibility test for the device 200 was conducted in the presence of 25 ppb concentration for 4 cycles. MOF-A/PDVT-10 device 200 showed a reproducibility with the same sensitivity levels for all the cycles.

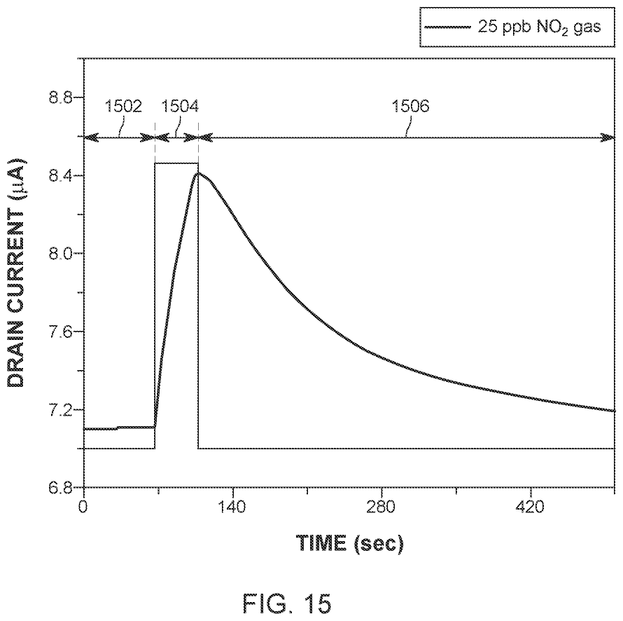

Next, as shown in FIG. 15, the response and recovery time values were obtained from the transient response of the MOF-A/PDVT-10 device 200 for 25 ppb NO₂ gas. The figure shows the saturation region 1502, the response region 1504, and the recovery region 1503. From the figure, the response and recovery times were measured to be around 43 sec and 438 sec, respectively. Bias stress, humidity, temperature, and ambient stability experiments were also conducted to study the impact of the various conditions on the NO₂ sensing performance. Continuous bias stress was applied at VD=VG=−20 V, and a gradual increase in the drain current of around 5 μA was observed at the end of 10 hours. During this experiment, the transfer drain current response was recorded every 2 hours. It was found that the MOF-A/PDVT-10 device 200 shows a negligible change in sensitivity due to the bias stress during exposure to 25 ppb NO₂ gas. Furthermore, the drain current was measured to be around 23 μA and 19 μA under 5% and 90% RH conditions, respectively. The sensitivity of the MOF-A/PDVT-10 device 200 when exposed to the humidity was calculated to be approximately 4.232 nA/% RH.

It is noted that a recent report in the field suggests that the PDVT-10 material is less stable in humid conditions. Thus, the MOF layer 120 that covers the entire top surface of the thin PDVT-10 layer 110 serves as a protection layer, and it has a significant role in improving the stability of the MOF-A/PDVT-10 device 200 against the humidity. This is due to the stability of the MOF-A in humid air, as has been proven by PXRD and sorption analysis. The hysteresis curves of the MOF-A/PDVT-10 device 200's response to 25 ppb NO₂ gas under different RH conditions from 30% to 90% have been calculated and the mean sensitivity along the absorption and desorption cycles was found to be around 22.715% and 20.435%, respectively, which has a negligible effect on the actual sensor's response to NO₂ gas (˜18%).

Further tests were conducted for the MOF-A/PDVT-10 device 200 when exposed to varying temperatures, and it was found that the sensitivity was drastically reduced with the increase in temperature. The sensitivity response logically decreases with the temperature increase, since in MOF-A, as in most other porous materials, the sorption equilibrium of gases decreases as the temperature rises. Hence, at temperatures around 100° C., negligible or no gas adsorption took place on the surface of the MOF-A/PDVT-10 device 200. It is believed that the interaction between the receptor layers and NO₂ gas was negligible at higher ambient temperatures. The MOF-A/PDVT-10 device 200 was also put to a shelf-life test over a period of 90 days in the laboratory. It was observed that the sensitivity of the device was quite stable over this period with insignificant changes in the drain current, thus avoiding any special storage requirements.

The gas sensing mechanism of the MOF-A/PDVT-10 device 200 is now discussed. As mentioned earlier, the pristine device 100 showed low sensitivity towards different analytes apart from NO₂. The reason behind its unique behavior was quantitatively probed using a KPFM system. The contact potential difference (CPD) of the polymer measured around −0.2325 eV in a normal ambient environment. The change in CPD (%) with respect to different analytes was determined and the response to NO₂ gas (5 ppm) was around 120% lower as compared to other analytes (at 100 ppm). This decreasing trend of CPD can be attributed to the increase in the surface electrons trapped by the adsorbed NO₂ gas, suggesting a dipole-dipole kind of interaction between the PDVT-10 polymer and NO₂ molecules. NO₂ is known to be an electrophile compound that makes it attract electrons to the single bond O in the structure (O═N—O). In contrast, an electron donor group (TVT) is available in the PDVT-10 material. After interacting with an electrophile like NO₂, it essentially induces more holes in the bulk of the OSC. In addition, the reaction tendency of NO₂ is strong compared to other oxidizing gases such as CO₂ and SO₂. Thus, the selectivity among oxidizing gases might be due to the presence of an extra electron in the orbital of N contributing to stronger withdrawing ability.

The role of MOF-A in enhancing the sensitivity and preserving the selectivity towards NO₂ was also studied. Firstly, the TPyP porphyrin ligand alone was deposited on the PDVT-10 material. This led to an enhancement in the sensitivity to all probed gases with this device, but no selectivity was achieved towards any of these gases. When constructing the MOF-A with the TPyP ligand, there was a small decrease in the sensitivity compared to the pure TPyP ligand, but the selectivity to NO₂ was preserved. This is due to the porosity of the MOF-A, which leads to better interaction with the NO₂ gas with confined space in the MOF-A framework compared to other gases. To validate whether this interaction was due to the interaction with the TiF₆ pillar only, MOF-B was deposited that had the same pillar as the MOF-A, but did not have the TPyP ligand on the PVDT10 (see FIG. 7). Then, the same sensing test was run using the MOF-B material, and the results showed no/slight enhancement in sensitivity. This proves that the enhancement in the sensitivity and the preservation of the selectivity were due to the synergy between the TPyP ligand and its presence in a confined space via its embedding in the MOF network. Thus, the proposed network selectively allows the target NO₂ molecules to pre-concentrate over the surface of the polymer to augment the overall sensitivity of MOF-A/PDVT-10 based device 200.

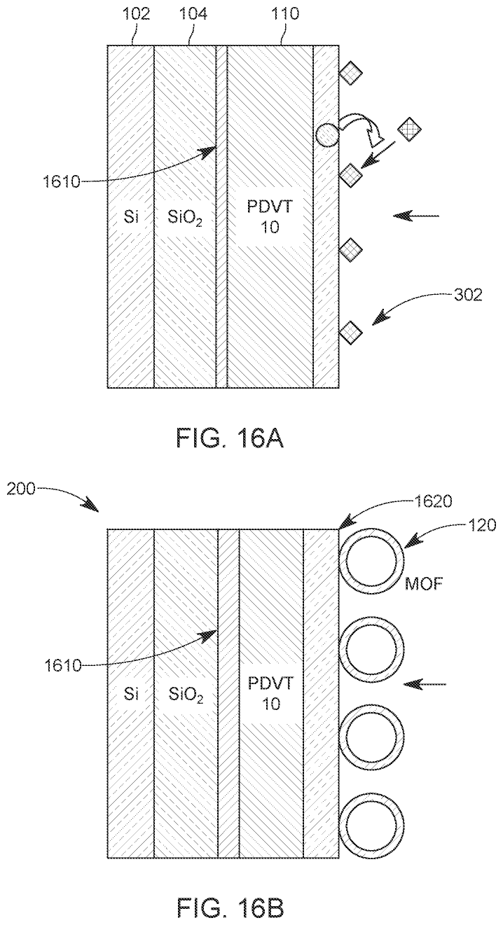

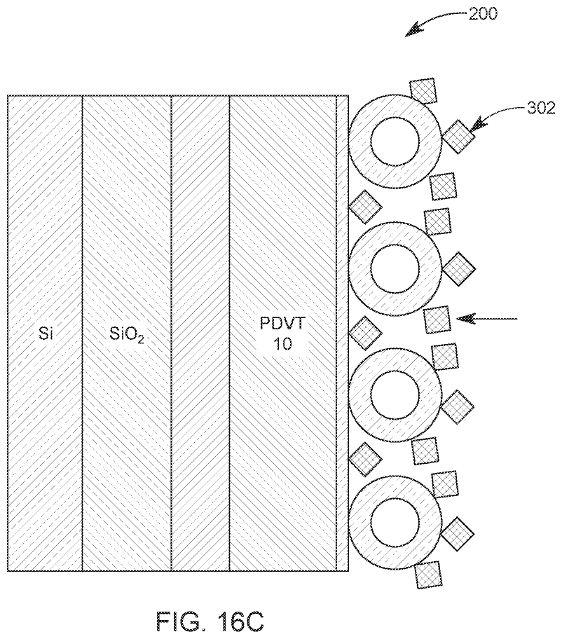

As discussed before, free holes were generated in the bulk region of the polymer PDVT-10 due to the physical adsorption of polar NO₂ molecules. These free holes tended to drift towards the dielectric/semiconductor interface region 1610, contributing to the increased channel current, as shown in FIG. 16A. Subsequently, to enhance the sensing properties, the MOF-A/PDVT-10 heterojunction OFET device 200 was tested. The corresponding energy band diagram is presented in FIG. 16B. Due to its high electron affinity, the MOF-A layer 120 tended to attract more electrons to a hole accumulation channel layer 1620 from the PDVT-10 layer 110. This helped in the increment of dominant free holes 1610 in the PDVT-10 bulk region. Due to the applied gate electric field, these additional free holes tended to drift towards the hole accumulation channel layer 1620 at the SiO₂/PDVT-10 interface, as shown in FIG. 16B. FIG. 16C displays the effect of the NO₂ gas interaction with the PDVT-10/MOF heterojunction structure 200.

To study the selectivity in these experiments, three oxidizing gases (SO₂, NO₂, and CO₂) were tested, and they came into contact with the device at the interface of MOF-A/PDVT-10. Using the KPFM, the extracted work function established the fact that the MOF-A is an n-type material, providing the scope for the formation of an electron-depleted space charge layer 1620 at the surface in the presence of the oxidizing gas. As is observed from the OFET data, there was an increase in the drain current due to further extraction of electrons (as compared to device 100) from the interface, leading to more hole doping in the channel region. Similarly, for reducing gases like NH₃, H₂S, and Hz, the opposite phenomenon took place, hence the evident slump in the drain current.

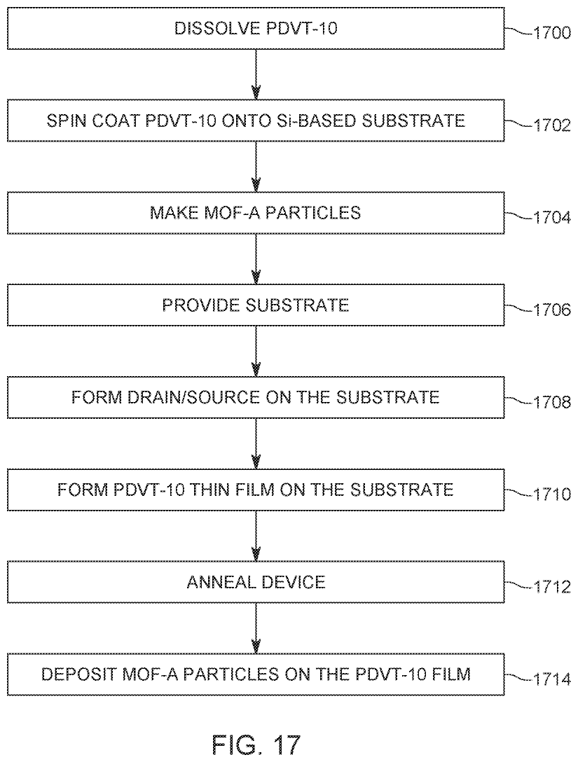

The steps of a method for making the MOF-A/PDVT-10 based device 200 are now discussed in more detail with regard to FIG. 17. The PDVT-10 material, Poly{3,6-dithiophen-2-yl-2,5-di(2-decyltetradecyl)-pyrrolo[3,4-c]pyrrole-1,4-dione-alt-thienylenevinylene-2,5-yl}(PDVT-10) organic semiconductor with molecular weight (M_{w})>30,000 was dissolved in step 1700 in Dichlorobenzene (DCB) organic solvent at a 3 mg/mL ratio. The prepared solution was stirred at 350 rpm for a period of 24 hours at 110 C. The PDVT-10 solution was spin coated in step 1702 on the surface of Si/SiO₂ or Quartz substrate for material characterization. After the film formation, some of the desired features, such as surface coverage, roughness, chemical composition, and electronic band gap properties, were characterized with the help of field enhanced scanning electron microscopy (FESEM), atomic force microscopy (AFM), Kelvin probe force microscopy (KPFM), Raman spectroscopy, photoelectron spectroscopy in air (PESA), and UV-Vis-NIR spectroscopy instruments. Based on these properties, the desired characteristics of the PDVT-10 material were selected and the associated spin coating conditions were stored for usage when the actual PDVT-10 layer 110 is formed in the device 200.

In step 1704, the MOF-A, i.e., [M′(M″F₆)(TPyP)]n particles generation was carried out under ultrasonic irradiation at a frequency of 40 KHz preset in an ultrasonic bath. The temperature was varied from 20 to 60° C. in all experiments. In this synthesis step, M′(NO₃)₂.xH₂O (0.04 mmol) and TPyP (12 mg, 0.02 mmol) are partially dissolved in 6 mL of DMF and 0.4 mL of 0.05M aqueous solution (NH₄)₂M″F₆ (0.02 mmol) were added into 20 mL scintillation vials. The vials were placed in the pre-heated ultrasonic bath. After the ultrasonic irradiation for a 4, 4, 10 h for a solution containing (NH₄)₂M″F₆ precursors, respectively, the products were isolated by centrifugation, and they were washed multiple times with DMF and activated with methanol.

Then, in step 1706, highly doped n-type (n++) silicon with thermally grown chlorinated SiO₂ layer wafers were used to fabricate bottom-gate bottom-contact (BGBC) OFET devices. The wafer samples were ultrasonically cleaned in acetone and isopropyl alcohol (IPA) solvents, for 5 minutes each. The cleaned samples were then rinsed in deionized (DI) water and blown under an N₂ gas flow for few seconds, and they were then dehumidified at 120° C. for 5 minutes. The source and drain interdigitated electrodes (IDE) 112 and 114, with a channel length and width of around 10 μm and 583640 μm, respectively, were deposited in step 1708 with radio frequency (RF) sputtered Ti (10 nm)/Au (100 nm) metals. It is noted that the ide patterns were formed using a standard photolithography process. The ide devices were used for the fabrication of both the pristine PDVT-10 device 100 and the MOF-A/PDVT-10 OFET device 200. The common feature in both devices was the formation in step 1710 of the PDVT-10 organic channel film 110 on the substrate 104. This film 110 was spin coated on the surface of the ide devices using the as-prepared 3 mg/mL PDVT-10 solution in step 1700. Subsequently, the PDVT-10 coated device was annealed in step 1712 by slowly increasing the temperature at a rate of 450° C./hr from 25° C. to 180° C. This high temperature was maintained for 5 min, and the device was then cooled down to room temperature. This resulted in the fabrication of the device 100. An additional step 1714 was used in the fabrication of the device 200. Step 1714 involved the deposition of MOF particles on the PDVT-10 film 110. To maintain consistency in the MOF film formation, a fixed quantity of 5 μL MOF solution was drop casted on the surface of the PDVT-10 film 110 to form the MOF-A layer 120. The MOF coated device 200 was annealed at 100° C. for 5 min to evaporate the residual solvents.

Thus, an ultrasensitive and highly selective OFET sensor 200 for NO2detection was obtained with the novel combination of the PDVT-10 material and the [Ni(TiF₆)(TPyP)]ₙ MOF-A layer 120. With the addition of the MOF layer, the sensitivity towards NO₂ analyte increased by 700%, and a negligible effect of humidity on the sensing performance was observed. The device 200 exhibits a high sensitivity of 680 nA/ppb with the synergistic combination of the PDVT-10 and MOF-A material in detecting NO₂, as compared to 7.6 nA/ppb (PDVT-10 alone). The device demonstrated reproducible performance from 8 ppb to 100 ppm, unaffected by humidity and ambient conditions. The sensor device was further subjected to relative humidity changes ranging from 5% to 90% to evaluate its performance in extreme conditions. Bias stress measurements conducted on the devices revealed a negligible effect on the gas sensing performance. Furthermore, it was observed that the device has a shelf-life larger than 2 months with insignificant changes in the baseline. Thus, this sensor can act as an alternative to existing sensor platforms due to its reduced complexity in fabrication and its high stability. These results additionally suggest that by choosing a proper synergistic combination of receptor materials, highly sensitive and selective sensors can be realized.

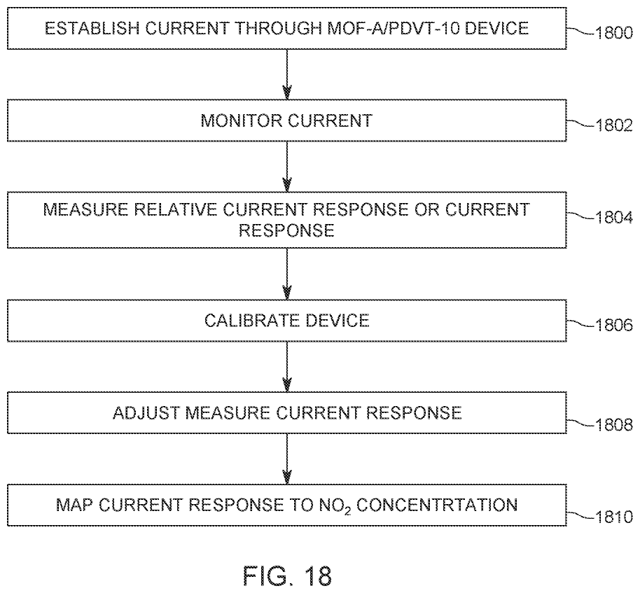

A method for measuring the NO₂ gas with the device 200 is now discussed with regard to FIG. 18. The sensor 200 is connected to a power source (V1, V2) that provides a gate voltage and a drain voltage, as schematically illustrated in FIG. 3. In step 1800, a current is established between the drain and source of the device 200. In step 1802, the current is monitored with the electronics 108. When the amount of NO₂ molecules is over the sensitivity threshold of the device, the relative change in the current response (see FIG. 13) or the current response (see FIG. 14) changes based on the NO₂ concentration. Thus, in step 1804, the electronics 108 monitors one of these two parameters, and maps the changes in the corresponding parameter to the corresponding NO₂ concentration. In optional step 1806, a calibration of the device 200 is performed and the results are stored in a memory of the electronics 108, and in step 1808, the electronics 108, which may also include a processor, adjusts the measured relative current response or current response based on the calibration results. Finally, in step 1810, the processor maps the measured relative current response or current response to the NO₂ concentration and provides the results to the user.

The disclosed embodiments provide a NO₂ detection device that uses a polymer semiconductor material as a channel and a metal-organic framework to coat the polymer semiconductor material and to enhance its selectivity. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] W. Wu, Y. Liu, D. Zhu, Chemical Society Reviews 2010, 39, 1489;
[2] Y. Zang, D. Huang, C. a. Di, D. Zhu, Advanced Materials 2016, 28, 4549;
[3] Q. Wang, S. Jiang, J. Qian, L. Song, L. Zhang, Y. Zhang, Y. Zhang, Y. Wang, X. Wang, Y. Shi, Scientific reports 2017, 7, 7830;
[4] O. Dalstein, D. R. Ceratti, C. Boissière, D. Grosso, A. Cattoni, M. Faustini, Advanced Functional Materials 2016, 26, 81;
[5] K. Chappanda, M. Tchalala, O. Shekhah, S. Surya, M. Eddaoudi, K. Salama, Sensors 2018, 18, 3898;
[6] J. Huang, M. Pei, H. S. Kim, H. Yang, D.-H. Hwang, Macromolecular Research 2019, 27, 227;
[7] J. Zhao, H. Lai, Z. Lyu, Y. Jiang, K. Xie, X. Wang, Q. Wu, L. Yang, Z. Jin, Y. Ma, Advanced materials 2015, 27, 3541;
[8] Y. Deng, Y. Chen, X. Zhang, H. Tian, C. Bao, D. Yan, Y. Geng, F. Wang, Macromolecules 2012, 45, 8621;
[9] H.-W. Lin, W.-Y. Lee, W.-C. Chen, Journal of Materials Chemistry 2012, 22, 2120;
[10] A. E. London, H. Chen, M. Sabuj, J. Tropp, M. Saghayezhian, N. Eedugurala, B. Zhang, Y. Liu, X. Gu, B. Wong, Science advances 2019, 5;
[10] G.-S. Ryu, K. H. Park, W.-T. Park, Y.-H. Kim, Y.-Y. Noh, Organic Electronics 2015, 23, 76;
[11] S. H. Yu, J. Cho, K. M. Sim, J. U. Ha, D. S. Chung, ACS applied materials & interfaces 2016, 8, 6570;
S. G. Surya, S. S. Nagarkar, S. K. Ghosh, P. Sonar, V. R. Rao, Sensors and Actuators B: Chemical 2016, 223, 114;
G.-S. Ryu, B. Nketia-Yawson, E.-Y. Choi, Y.-Y. Noh, Organic Electronics 2017, 51, 264;
Y. Yang, G. Zhang, H. Luo, J. Yao, Z. Liu, D. Zhang, ACS applied materials & interfaces 2015, 8, 3635;
K. N. Chappanda, A. Chaix, S. G. Surya, B. A. Moosa, N. M. Khashab, K. N. Salama, Sensors and Actuators B: Chemical 2019, 294, 40;
S. M. Tawfik, M. Sharipov, S. Kakhkhorov, M. R. Elmasry, Y. I. Lee, Advanced Science 2019, 6, 1801467;
A. Chaix, G. Mouchaham, A. Shkurenko, P. Hoang, B. Moosa, P. M. Bhatt, K. Adil, K. N. Salama, M. Eddaoudi, N. M. Khashab, Journal of the American Chemical Society 2018, 140, 14571;
Q. Lin, C. Mao, A. Kong, X. Bu, X. Zhao, P. Feng, Journal of Materials Chemistry A 2017, 5, 21189;
V.-Chernikova, MOFs exploration: from synthesis and thin film fabrication to separation and sensing applications, 2018;
C. Sapsanis, H. Omran, V. Chernikova, O. Shekhah, Y. Belmabkhout, U. Buttner, M. Eddaoudi, K. Salama, Sensors 2015, 15, 18153.

1. An NO₂ detection device comprising:
a substrate;
a drain formed on the substrate;
a source formed on the substrate;
a p-type polymer semiconductor layer formed on the substrate, between the drain and the source; and
an n-type metal-organic framework layer located over the p-type polymer semiconductor layer,
wherein the n-type metal-organic framework layer has apertures having a size larger than a size of the NO₂ molecules so that the NO₂ molecules pass through the n-type metal-organic framework layer to arrive at the p-type polymer semiconductor layer to increase an electrical current.

2. The device of claim 1, wherein the p-type polymer semiconductor layer includes a Diketopyrrolopyrrole (DPP) copolymer having thiophene donor blocks.

3. The device of claim 2, wherein the n-type metal-organic framework layer includes [M′₂L₂(M″F₆)]ₙ, where M′ is a metal with octahedral geometry, L is ditopic nitrogen containing linker, and (M″F₆) is an inorganic pillar.

4. The device of claim 3, wherein M′ is Ni and M″ is Ti.

5. The device of claim 2, wherein the n-type metal-organic framework layer includes [Ni(TPyP)(TiF₆)]ₙ, where TPyP is 5,10,15,20-Tetra(4-pyridyl)porphyrin.

6. The device of claim 1, wherein a distance between the drain and source is about 10 μm.

7. The device of claim 1, wherein a thickness of the p-type polymer semiconductor layer is between 15 and 70 nm, and a thickness of the n-type metal-organic framework layer is between 10 and 100 nm.

8. The device of claim 1, wherein an entire top surface of the p-type polymer semiconductor layer is coated by the n-type metal-organic framework layer.

9. The device of claim 1, wherein the current response is substantially proportional to the NO₂ concentration.

10. An n-type metal-organic framework material comprising:
[M′₂L₂(M″F₆)]ₙ,
wherein M′ is a metal with octahedral geometry, L is ditopic nitrogen containing linker, and (M″F₆) is an inorganic pillar.

11. The material of claim 10, wherein M′ is Ni and M″ is Ti.

12. The material of claim 11, wherein L is 5,10,15,20-Tetra(4-pyridyl)porphyrin.

13. The material of claim 10, wherein the material has apertures having a size larger than a size of the NO₂ molecules so that the NO₂ molecules pass through the material.

14. A method of making an NO₂ detection device, the method comprising:
dissolving a p-type polymer semiconductor material (PDVT-10) into a solvent;
generating an n-type metal-organic framework material (MOF-A);
providing a substrate based on Si;
forming a drain and a source on the substrate;
depositing the p-type polymer semiconductor material (PDVT-10) onto the substrate, between the drain and the source, to form a polymer semiconductor layer; and
depositing the n-type metal-organic framework material (MOF-A) onto the polymer semiconductor layer to form an n-type metal-organic framework layer,
wherein the n-type metal-organic framework layer has apertures having a size larger than a size of the NO₂ molecules so that the NO₂ molecules pass through the n-type metal-organic framework layer to arrive at the p-type polymer semiconductor layer to increase an electrical current.

15. The method of claim 14, wherein the p-type polymer semiconductor layer includes a Diketopyrrolopyrrole (DPP) copolymer having thiophene donor blocks.

16. The method of claim 15, wherein the n-type metal-organic framework layer includes [M′₂L₂(M″F₆)]ₙ, where M′ is a metal with octahedral geometry, L is ditopic nitrogen containing linker, and (M″F₆) is an inorganic pillar.

17. The method of claim 16, wherein M′ is Ni and M″ is Ti.

18. The method of claim 14, wherein the n-type metal-organic framework layer includes [Ni(TPyP)(TiF₆)]ₙ, where TPyP is 5,10,15,20-Tetra(4-pyridyl)porphyrin.

19. The method of claim 14, wherein a distance between the drain and source is about 10 μm.

20. The method of claim 14, wherein a thickness of the p-type polymer semiconductor layer is between 15 and 70 nm, and a thickness of the n-type metal-organic framework layer is between 10 and 100 nm.
